(19) United States
(12) Patent Application Publication
(10) Pub. No.: US 2010/0035762 A1
(43) Pub. Date: Feb. 11, 2010

(54) Prognostic Methods in Colorectal Cancer

(75) Inventors: Simo Schwartz Navarro, Cardedeu (ES); Diego Arango del Corro, Barcelona (ES); Lauri A. Aaltonen, Espoo (FI); John M. Mariadason, Victoria (AU); Carlos Buesa Arjol, Barcelona (ES)

Correspondence Address: TRASKBRITT, P.C., P.O. BOX 2550, SALT LAKE CITY, UT, 84110, US

(73) Assignee: Oryzon Genomics, S.A., Barcelona (ES)

(21) Appl. No.: 12/308,808

(22) PCT Filed: Jun. 21, 2007
(86) PCT No.: PCT/EP2007/056222

§ 371 Date: Oct. 19, 2009

(30) Foreign Application Priority Data
Jun. 22, 2006 (ES) P 200601719

(51) Int. Cl. C40B 30/04; C12Q 1/68; G01N 33/53; B01D 57/02; G01N 33/559; G01N 33/561

(52) U.S. Cl. 506/9; 435/6; 435/7.1; 204/461

(57) ABSTRACT

Prognostic method in colorectal cancer based on the determination of the expression levels of the EphB4 gene in tumours of patients afflicted by this disease. The levels can be used as a marker for the probability of the recurrence of the cancer in the patient and for the prognostics of the sensitivity that the tumours present to treatment with 5-fluorouracil, allowing to establish the more adequate therapeutic strategy for every patient.

### FIELD OF THE INVENTION

The present invention refers to a method to determine the prognostic of colorectal cancer based on the relationship between the expression level of the EphB4 gene and the degree of sensibility of a tumour to treatment with 5-fluorouracil. The method permits the identification of patients with tumours that have a low probability to respond to treatment with or 5-fluorouracil, and can be used as a prognostic criterion for these tumours and as a means to select the treatment thereof.

### BACKGROUND ART

Colorectal cancer is one of the more common types of cancer in the European Union. In the year 2000, a total 220.000 new cases were detected and 120.000 deaths were registered. In addition, both the incidence and the mortality of the type of cancer have raised (12.4% and 6.1% respectively, from 1995 to 2000).

The surgical removal of the principal tumour is the most used treatment for patients with stage II and III colorectal cancer (locally advanced tumors without distant metastasis). This treatment results in the cure of approximately 40-50% of these patients. Nevertheless, the remaining 50-60% present micrometastases that result in the recurrence of the disease. During the last decade, numerous studies have demonstrated that the use of adjuvant therapy with 5-fluorouracil (5FU) after surgical resection results in a significant reduction of the number of patients with recurrence and an increase in survival of these patients (Augenlicht et al., (1997) Low-level c-myc amplification in human colonic carcinoma cell lines and tumors: a frequent, p53-independent mutation associated with improved outcome in a randomized multi-institutional trial. Cancer Res, 57, 1769-75; Moertel et al., (1990) Levamisole and fluorouracil for adjuvant therapy of surgically removed colon carcinoma. N Engl J Med, 322, 352-8; Moertel et al., (1995) Fluorouracil plus levamisole as effective adjuvant therapy after resection of stage III colon carcinoma: a final report. Ann Intern Med, 122, 321-6).

Since actually no way exists to identify the patients that may benefit from this treatment, all patients in stage III and a significant percentage of patients in stage II are treated with adjuvant therapy with 5FU. Nevertheless, approximately 80% of the patients will not benefit from this treatment, either because they were already cured by surgery or because the tumour is resistant to 5-fluorouracil, and thus, the standard treatment is far from ideal for these patients.

During more than 4 decades, the only chemotherapeutic agent with demonstrated efficacy against colorectal cancer was 5FU. Recently, other agents like camptothecin and oxaliplatin have shown efficacy against colorectal cancer (Arango & Augenlicht, (2001) New approaches to colorectal cancer treatment. In Recent Research Developments in Cancer, Vol. 3. pp. 385-395. Transworld Research Network: Trivandrum; Cunningham et al., (1998) Randomised trial of irinotecan plus supportive care versus supportive care alone after fluorouracil failure for patients with metastatic colorectal cancer. The Lancet, 352, 1413-1418; de Gramont et al., (1997) Oxaliplatin with high-dose leucovorin and 5-fluorouracil 48-hour continuous infusion in pretreated metastatic colorectal cancer. Eur J Cancer, 33, 214-9). The availability of these alternative agents makes evident the necessity to identify patients and stratify the patients with respect to their probability of response to the treatment with 5FU. This will permit the optimization of the chemotherapeutic treatment of patients with locally advanced colorectal cancer and it will permit to improve their survival through administration of additional or alternative chemotherapeutic agents.

The receptors EPH (erythropoietin-producing hepatocellular) and their ligands, the Ephrins (EPHNs), constitute a major family of receptors with known tyrosine kinase. EPHs and Ephrins are capable to transmit extracellular signals and to modulate migration and cellular adhesion (Brantley-Sieders et al., (2004) Eph receptor tyrosine kinases in tumor and tumor microenvironment. Curr Pharm Des, 10, 343142). In the past, aberrant expression of various members of this family of tyrosine kinases was observed in tumour tissues, and recent publications indicate an important role of these genes in colorectal tumour formation (Alazzouzi et al., (2005) Mechanisms of inactivation of the receptor tyrosine kinase EPHB2 in colorectal tumors. Cancer Research, 65, 10170-10173; Hafner et al., (2004) Differential gene expression of Eph receptors and ephrins in benign human tissues and cancers. Clin Chem, 50, 490-9; Liu et al., (2002) Coexpression of ephrin-Bs and their receptors in colon carcinoma. Cancer, 94, 934-9; Liu et al., (2004) Effects of overexpression of ephrin-B2 on tumour growth in human colorectal cancer. Br J Cancer, 90, 1620-6; Oba et al., (2001) Genomic structure and loss of heterozygosity of EphB2 in colorectal cancer. Cancer Lett, 164, 97-104).

The EPH receptors are integral membrane proteins that possess an extracellular region that mediates the union of the ligand with an highly conserved N terminal domain, which is followed by a cystein rich region and two fibronectin type III repeats, that are essential for dimerization and interaction with other proteins (Labrador et al., (1997) The N-terminal globular domain of Eph receptors is sufficient for ligand binding and receptor signaling. Embo J, 16, 3889-97). The intracellular portion of these receptors contain a yuxtamembrane region, a conserved kinase domain, a SAM motif and a union domain to PDZ (Kalo & Pasquale, (1999) Signal transfer by eph receptors. Cell Tissue Res, 298, 1-9). The human genome contains at least 15 EPH receptors (Gale et al., (1996) Eph receptors and ligands comprise two major specificity subclasses and are reciprocally compartmentalized during embryogenesis. Neuron, 17, 9-19). The ligands of these receptors (ephrins) are linked to the cellular membrane. In accordance with the Committee of Nomenclature for these proteins (Committee, E.N. (1997) Unified nomenclature for Eph family receptors and their ligands, the. ephrins. Eph Nomenclature Committee. Cell, 90, 403-4), the ephrins are divided in two structural subtypes according to their anchorage to the membrane; which can be through a GPI (“Glycosyl Phosphatidyl Inositol”) union or through a transmembrane domain (subclasses A and B, respectively). Up to date, six A class ligands and three B class ligands have been identified.

The signaling cascade begins with the union of EPH and Ephrins localized in opposite cellular surfaces. This inicial high affinity dimerization leads to the grouping of these dimers and formation of heterotetramers that, in their turn, are able to form higher order complexes. The heterodimerization of EPH-Ephrin results in autophosphorilation of the EPH receptor in various tyrosine residues, leading in its turn to the increase of the receptor kinase activity and in the creation of coupling sites for signaling molecules. The dimerization of EPH-Ephrin promotes the association of these receptors with other adaptor molecules independent of their autophosphorylation and can modulate the pathways of Ras and Rho. In addition to the signaling started by the EPH, the ligands are also capable to initiate a reverse signaling. Consequently, the union of a cell expressing EPH receptors and a cell expressing a compatible Ephrin can generate a bidirectional signal in both cells.

The EPH-Ephrin system regulates various important cellular processes. Through the small GTPases of the Ras and Rho family, the EPH-Ephrin system regulates the structure of the cytoskeleton (Noren & Pasquale, (2004) Eph receptor-ephrin bidirectional signals that target Ras and Rho proteins. Cell Signal, 16, 655-66; Zou et al., (1999) An Eph receptor regulates integrin activity through R-Ras. Proc Natl Acad Sci U S A, 96, 13813-8). The signals released after the activation of the EPH-Ephrin system regulate the attraction and repulsion forces between cells of different tissues. These signals are important in the organization of the vasculature and nervous system. (Altick et al., (2005) EphB receptor tyrosine kinases control morphological development of the ventral midbrain. Mech Dev, 122, 501-12; Noren et al., (2004) Interplay between EphB4 on tumor cells and vascular ephrin-B2 regulates tumor growth. Proc Natl Acad Sci U S A, 101, 5583-8).

Recently the important role of various EPH and Ephrins in the maintenance of the structure and cellular migration of the intestinal crypts was demonstrated (Batile et al., (2002) Beta-catenin and TCF mediate cell positioning in the intestinal epithelium by controlling the expression of EphB/ephrinB. Cell, 111, 251-63; van de Wetering et al., (2002) The beta-catenin/TCF4 complex imposes a crypt progenitor phenotype on colorectal cancer cells. Cell, 111, 241-50).

Mutations in the tumour supressor gene APC are the most frequent genetic alterations in colorectal cancer. The normal function of APC is to facilitate the labelling of β-catenin for destruction in a multiproteic complex including GSK3-β and Axin. Nevertheless, mutations in APC (or other members of the complex) result in the accumulation and nuclear translocation of β-catenin. In the nucleus, β-catenin binds transcription factors of the TCF family, leading to the transcriptional activation of a large number of genes, some of which have a strong oncogenic activity, like Cyclin D and the transcription factor c-Myc (He et al., (1998) Identification of c-MYC as a target of the APC pathway. Science, 281, 1509-12). In a recent study of the group of Dr. Clevers (Batlle et al., (2002) Beta-catenin and TCF mediate cell positioning in the intestinal epithelium by controlling the expression of EphB/ephrinB. Cell, 111, 251-63) it was shown that the β-catenin/TCF system regulates the expression level of various members of the EPH/Ephrin system (EPHB2, EPHB3 and Ephin-B1). Animals deficient in EPHB2 and/or EPHB3 display aberrant cellular migration patterns in the intestinal crypts, and intestinal tumours caused by mutations in APC in a murine model, display high levels of expression of EPHB2 (Batlle et al., (2002) Beta-catenin and TCF mediate cell positioning in the intestinal epithelium by controlling the expression of EphB/ephrinB. Cell, 111, 251-63). In addition, a recent study has demonstrated that the lack of EPHB2 is important in the progression of gastrointestinal tumours initiated by mutations in APC, both in model animals and in human tumours (Batlle et al., (2005) EphB receptor activity suppresses colorectal cancer progression. Nature, 435, 1126-30).

An important part of colorectal tumours have defects in the repair system of mismatched bases, resulting in an increase in the number of mutations in tumour cells. This effect is noted particularly in regions of the genome with repetitions of mono or dinucleotides called microsatellites. This phenotype is known as MSI (“microsatellite instable”) and results in the selection of tumour cells that acquire mutations in microsatellites in the coding sequences of key genes in the initiation of tumour initiation or progression like TGF-β receptor II or BAX. Previous studies have found mutations in 52,2% (71/136) of the colorectal carcinomas with an MSI phenotype in microsatellite A9 in Exon 17 of EPHB2, one of the receptors that regulates the positioning of the cells in the intestinal epithelium and whose functional importance in the progression of colorectal tumours has recently been demonstrated. Also, in tumours without defects in the base repair system (MSS; microsatellite stable), 52,5% (21/41) display hypermethylation of CpG islands in the EPHB2 promoter and 40% (8/21) suffer from loss of heterozigosity (LOH) in chromosome 1p36, the genomic region in which EPHB2 is localized. The hypermethylation and chromosomal deletions are epigenetic mechanisms that explain the reduction of EPHB2 observed in colorectal tumours compared to normal epithelia. Also, in a recent study it was shown that the low levels of EPHB2 is one of the molecular factors that identifies a new type of colorectal tumours with serrated histology (“serrated tumours”).

EPHs and Ephrins regulate the adherence and cellular movement through signaling cascades like Ras and Rho (Elowe et al., (2001) Downregulation of the Ras-mitogen-activated protein kinase pathway by the EphB2 receptor tyrosine kinase is required for ephrin-induced neurite retraction. Mol Cell Biol, 21, 7429-41; Tanaka et al., (2003). Association of Dishevelled with Eph tyrosine kinase receptor and ephrin mediates cell repulsion. Embo J, 22, 847-58; Zou et al., (1999) An Eph receptor regulates integrin activity through R-Ras. Proc Natl Acad Sci U S A, 96, 13813-8). In corcordance with this observation, recently, low levels of expression of RHOA in colorectal tumours have been associated to a significant reduction (p=0,01) in the survival of these patients (Arango et al., (2005) Utilization of microarray analysis to predict prognosis of Dukes C colorectal cancer patients. Gastroenterology, 129, 874-884).

Actually, there exists a great need to find new pronostic markers and markers that predict the response to treatments for patients with colorectal cancer. The increasing number of markers that have demonstrated their utility in studies have not been translated in rutinary clinical applications yet, (Alazzouzi et al., (2005) SMAD4 as a prognostic marker in colorectal cancer. Clin Cancer Res, 11, 2606-2611; Alhopuro et al., (2005) SMAD4 levels and response to 5-fluorouracil in colorectal cancer. Clin Cancer Res. Sep 1;11(17):6311-6; Arango et al., (2001) c-myc/p53 interaction determines sensitivity of human colon carcinoma cells to 5-fluorouracil in vitro and in vivo. Cancer Res, 61, 4910-5; Arango et al., (2005) Utilization of microarray analysis to predict prognosis of Dukes C colorectal cancer patients. Gastroenterology, 129, 874-884; Arango et al., (2003) c-Myc overexpression sensitizes colon cancer cells to camptothecin-induced apoptosis. British Journal of Cancer, 89, 1757-65), and patients with colorectal cancer continue to receive a common and standard treatment, irrespective of the great heterogeneity demonstrated in their pronostic and response to the treatment. Previous studies have found deregulation in the levels of expression of other members of the family of EPH receptors (Martiny-Baron et al., (2004) Inhibition of tumor growth and angiogenesis by soluble EphB4. Neoplasia, 6, 248-57; Wu et al., (2004) Expression of Ephb2 and Ephb4 in breast carcinoma. Pathol Oncol Res, 10, 26-33; Xia et al., (2005) EphB4 expression and biological significance in prostate cancer. Cancer Res, 65, 4623-32).

### DESCRIPTION OF THE INVENTION

According to the present invention, EPHB4 is a good pronostic marker and a marker for the response to the treatment with 5FU for patients with colorectal cancer, in a way that the expression levels of EPHB4 in colorectal tumours can be used to predict the probability of recurrence and survival of these patients. Low levels of EPHB4 expression allow the identification of a group of patients with a mean survival time of 1,8 years, compared to a group with high levels of EPHB4 that will have a mean survival time of over 9 years. It is important to stress that these results were obtained from data of two independent studies. Patients with tumours with low levels of EPHB4 have a bad prognostic and response to 5FU treatment. Consequently, the analysis of the EPHB4 levels in biopsies or after surgical resection of the tumour, permit the identification of patients with a low probability to respond to the standard treatment (surgery plus 5FU), for which a more aggressive treatment including chemotherapy with oxaliplatin and/or camptothecin would be indicated.

An object of the present invention relates to a prognostic method in colorectal cancer that comprises the determination of the expression level of EphB4 in a biological sample isolated from a patient, where the expression level is used as a prognostic marker and as a marker for the response of the colorectal tumour to treatment with 5-fluorouracil.

Particularly, low levels of expression of the gene are indicative of a bad prognostic and low sensitivity of the colorectal tumour to treatment with 5-fluorouracil, while high levels of expression are indicative of a good prognosis and high sensitivity to the treatment.

In one embodiment of the invention, the sample analyzed can be the RNA encoding EphB4 or fragments of this RNA, and can be isolated from cells obtained through biopsy or any other method of extraction. Preferably, the determination can be carried out through amplification through PCR, SDA or any other method for amplification of nucleic acids. Alternatively, the determination can be carried out using DNA microarrays prepared through deposition of oligos or through synthesis in situ by photolithography or any other mechanism. Alternatively, the determination is carried out through in situ hybridization using probes labelled through any kind of labelling method. Alternatively, the determination is carried out through gel electrophoresis, wherein electrophoresis can be optionally carried out through transfer to a membrane and hybridization with a specific probe. Alternatively, the determination is carried out through NMR or any other technique for diagnostics through image analysis. Alternatively, the determination is carried out through NMR or any other technique for diagnostics through image analysis and the use of paramagnetic nanoparticles or any other type of detectable nanoparticles functionalized with antibodies or any other means.

In another embodiment of the invention, the sample analyzed can be the protein encoded by the gene or fragments thereof. Preferably, the determination is carried out through the incubation with a specific antibody. Optionally, the determination can be carried out through Western blot or immunohistochemistry. Alternatively, the determination is carried out through protein gel electrophoresis. Alternatively, the determination is carried out through protein microarrays. Alternatively, the determination is carried out through ELISA or any other enzymatic method. Alternatively, the determination is carried out through NMR or any other technique for diagnostics through image analysis. Alternatively, the determination is carried out through NMR or any other technique for diagnostics through image analysis by using paramagnetic nanoparticles or any other type of detectable nanoparticles functionalized with antibodies or any other means.

A further object of the present invention relates to a kit to apply the prognostic method for colorectal cancer that comprises reagents and additives appropriate to determine the expression level of the gene EphB4.

The present invention also has as an object a kit that comprises a specific anti-EPHB4 antibody, a secondary antibody that can be conjugated to the primary antibody, a visualization reagent, a chromogenic substance and other reagents and additives necessary to determine the expression of the EphB4 gene in tissue sections.

In one embodiment of the invention, the kit includes positive and negative controls that permit the quantification of the expression level of EphB4. This quantification can be carried out manually or in automated form.

Another object of the present invention relates to a method to analyse compounds with therapeutic potential in colorectal cancer that comprise determining the capacity of these compounds to increase the expression level of the EphB4 gene.

Also, the present invention relates to a pharmaceutical composition that comprises an effective amount of the compounds with therapeutic potential according to the method described above, and one or more pharmaceutically acceptable excipients.

On the other hand, the present invention also relates to the use of compounds with therapeutic potential obtained according to the method described above for the preparation of a medicament for the treatment or prevention of colorectal cancer or the pre malignant condition thereof. As used therein the term “treatment” includes treatment and management of such condition, as well as prevention against new tumours.

In the present invention, the assignment of the distinctive threshold between high and low level depends on the technique used to determine the expression level of EphB4. Thus, for example, employing a semi quantitative analysis using immunohistochemistry, a threshold value of 2 was assigned in a scale of 0 to 4.

The studies performed using immunohistochemistry (IHQ) on tissue sections from colorectal cancer with specific antibodies anti-EPHB4, demonstrated a clear gradient in the expression of EPHB4 in the epithelium of the colon, with a maximal expression in the depth of the crypts and reduced expression in the luminal zone (FIG. 1., 1-2). High variability was detected in the expression levels of EPHB4 in colorectal tumours (FIG. 1, 4-8).

In addition, the levels of EPHB4 expression were studied in a collection of 137 human colorectal tumours by means of IHQ in sections of a tissue microarray (Table 1). It was observed that patients with tumours with low EPHB4 expression levels have a time of survival which is significantly lower (p=0,009) (FIG. 2a). When EPHB4 was considered as an independent variable, a significant association was observed between low levels of EPHB4 and total survival or disease free time (Cox Regression p<0.05). Also, in a multivariate analysis including sex, age and tumour localization (colon or rectum), histological grade and EPHB4 levels, these continued being a strong prognostic marker for total survival and disease free time (Cox Regression p=0,005 and p=0,009 respectively). These results were confirmed independently using a second collection of 125 colorectal tumours (Table 2 and FIG. 2b). In addition, the expression levels of EPHB4 were studied in 16 metastasis and in lymphatic glands of another 16 patients with colorectal cancer; and in these metastases, lower EPHB4 levels were detected than in the primary tumours (t-test; p=0,027), reinforcing the idea that low EPHB4 levels are an indications of a bad prognostic in those patients. No associations were detected between EPHB4 levels and other clinicopathological variables (Tables 1 and 2).

TABLE 1
Clinicopatological Factors of 137 Dukes C patients in the study.

| | Total | High EPHB4 | Low EPHB4 | p |
|---|---|---|---|---|
| Sex, n (%) | | | | |
| Female | 69 (50.4) | 15 (40.5) | 54 (54) | |
| Male | 68 (49.6) | 22 (59.5) | 46 (46) | 0.16¹ |
| Age (years), | 67.5 ± 12.0 | 70.4 ± 11.7 | 64.9 ± 11.8 | 0.16² |
| mean ± SD | | | | |
| Localization, n (%) | | | | |
| Colon | 75 (55.1) | 19 (51.4) | 56 (56.6) | |
| Rectum | 61 (44.9) | 18 (48.6) | 43 (43.4) | 0.59¹ |
| Grade of | | | | |
| differentiation, n (%) | | | | |
| High | 19 (14.1) | 4 (10.8) | 15 (15.3) | |
| Medium | 103 (76.3) | 31 (83.8) | 72 (73.5) | |
| Low | 13 (9.6) | 2 (5.4) | 11 (11.2) | 0.43¹ |
| Survival at | | | | |
| 5 years, n (%) | | | | |
| alive | 74 (54) | 22 (59.5) | 41 (41) | |
| death | 63 (46) | 15 (40.5) | 59 (59) | 0.05¹ |

¹Chi-square test and
²Mann Whitney test

TABLE 2
Clinicopathological Factors of the 125 Dukes C patients
studied in the second confirmatory experiment

| | Total | High EPHB4 | Low EPHB4 | p |
|---|---|---|---|---|
| Sex, n (%) | | | | |
| Female | 80 (64) | 32 (58.2) | 48 (68.6) | |
| Male | 45 (36) | 23 (41.8) | 22 (31.4) | 0.23¹ |
| Age (years), | 66.9 ± 12.2 | 66.8 ± 13.7 | 67 ± 11.1 | 0.16² |
| mean ± SD | | | | |
| Grade of | | | | |
| differentiation, n (%) | | | | |
| High/medium | 109 (90.8) | 48 (96) | 61 (87.1) | |
| Low | 11 (9.2) | 2 (4) | 9 (12.9) | 0.12³ |
| Dukes, n (%) | | | | |
| A | 7 (5.6) | 5 (9.1) | 2 (2.9) | |
| B | 56 (44.8) | 27 (49.1) | 29 (41.4) | |
| C | 45 (36) | 19 (34.5) | 26 (37.1) | |
| D | 17 (13.6) | 4 (7.3) | 13 (18.6) | 0.14¹ |

¹Chi-square test;
²Mann Whitney test and
³Fisher's exact test

Together, these data demonstrate that EPHB4 levels can be used as a prognostic marker and as a marker to predict response to the standard treatment in patients with colorectal cancer. The marker can be employed clinically to identify patients with a bad prognostic when treated with the standard therapy, and which could benefit from a more aggressive treatment.

To demonstrate a possible function of EPHB4 in colorectal tumour, we reintroduced EPHB4 in a colorectal cancer cell line that does not express the gene (SW837; FIG. 3A). Stable transfection of these cells with an expression vector resulted in a significative reduction of the number of cells with long term clonogenic capacity in comparison to the cultured cells transfected with the empty vector (FIG. 3B). These experiments demonstrate not only that EPHB4 can be used as a prognostic marker for patients with colorectal cancer, it also shows that EPHB4 has an important role in the oncogenic process in this organ.

Consequently, the present invention demonstrates a correlation between the levels of EPHB4 and the probability of recurrence in patients with colorectal cancer (with and without treatment with 5-fluorouracil) whatever its stage of development is, meaning that we now have a novel molecular tool that permits to determine the prognostics of the disease and to select the most fit treatment for the patient, which was not possible with the previously available methods.

The present invention also demonstrates a pharmaceutical composition that comprises an effective amount of a compound with therapeutic potential. Specifically, it was demonstrated that EphB4 is frequently methylated in colorectal cancer; and that 5-aza-cytidin reduces the methylation and induces of expression of EphB4. Given the correlation of the level of expression of EphB4 and the pronostic, the treatment with 5-aza-cytidin or other compounds capable of increasing the expression of this gene, can modify the prognostic of colon cancer and the response of the tumor to treatment with 5-fluorouracil.

### SHORT DESCRIPTION OF THE FIGURES

FIG. 1: EPHB4 expression levels in colorectal tissues. The immunohistochemical staining with an anti-EPHB4 antibody shows a gradient of expression in the normal colon epithelium, with maximal levels in the center of the crypts (1-2). EPHB4 is located mainly in the cytoplasmic membrane in most of the tumours analyzed (3). A high variability was observed in the expression level of EPHB4 in the 137 colorectal-tumours investigated (4-8).

FIG. 2: low levels of EPHB4 in tumours are associated with a bad prognostic in patients with colorectal cancer. A) Total survival curves (Kaplan-Mayer) and survival without recurrence in 137 patients with colorectal cancer in Dukes stage C. Patients with low levels of EPHB4 have a survival time which is significantly shorter than patients with tumours with high levels of EPHB4 (Log-rank test p<0.01). B) This result was independently confirmed in a group of 125 different patients with colorectal cancer Dukes stage A-D. Patients with low EPHB4 expression levels in tumours have a worse prognostic than patients with tumours with high EPHB4 levels (Log-rank test p=0.02).

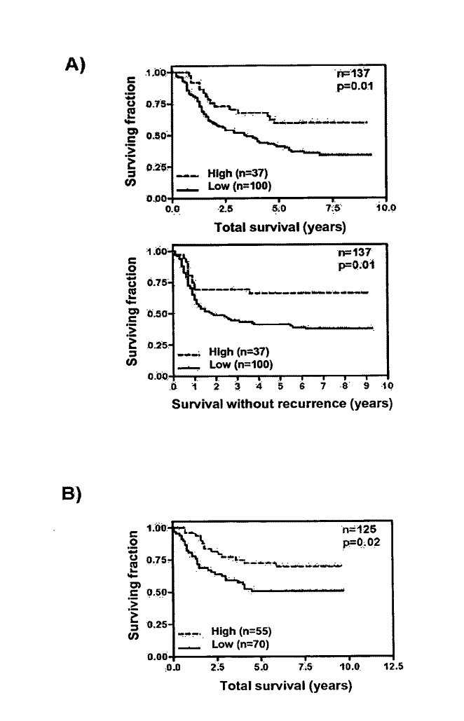

FIG. 3: Survival Curve (Kaplan Mayer) representing the time free of disease. The levels of EPHB4 were quantified by image analysis after histological staining with the anti EPHB4 antibody. The fractionated area was calculated and high EPHB4 expression was defined for cases in which the fractionated area was superior to 50%. Patients with low levels of EPHB4 expression in tumours have a worse prognostic than patients with high EPHB4 expression levels with a high statistical significance (P value 0,0065).

FIG. 4: the reintroduction of EPHB4 in colorectal tumour cells results in a reduction of their clonogenic potential. A) A high variability in the levels of EPHB4 as determined by Western blotting was detected in colorectal tumour cell lines. The levels of beta-actin were used as a loading control. B) The transfection of SW837 cells with an expression vector of EPHB4 (pcDNA-EPHB4) resulted in a significant reduction in the number of colonies resistent to neomycin after 14 days of selection. Representative results of colonies stained with crystal violet are shown in the inferior panel. The histogram in the superior part shows the result of the quantification of the number of colonies observed in 3 independent experiments (mean±Standard deviation; *t-test p=0.0008).

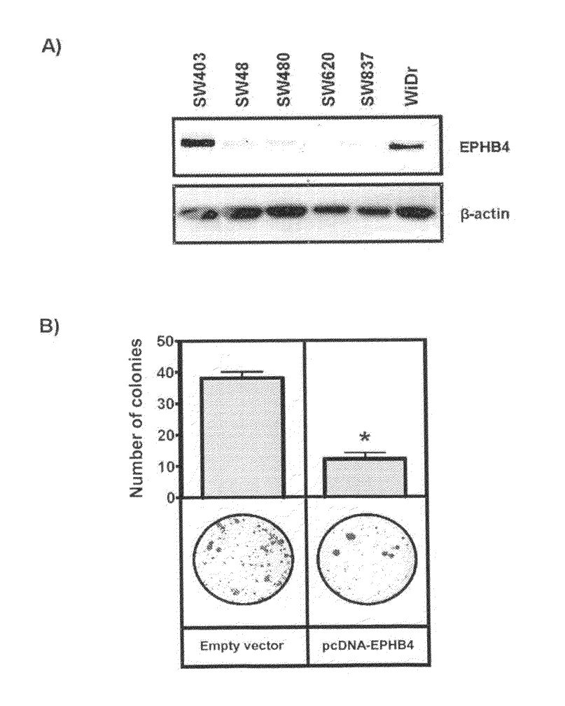

FIG. 5: The treatment of the colorectal cancer cell line SW620 with 5-aza-cytidine reduces the methylation of the EphB4 promoter and induces its gene expression. Top panel: changes in gene expression a measured by RT-PCR. Lower panel: changes in methylation a measure by PCR on bisulfite treated DNA with methylation specific primers.

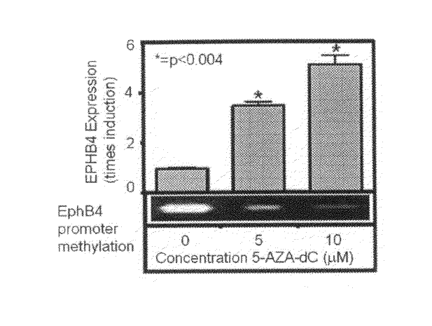

### EXAMPLES

In the following, some non limiting examples of the invention are described.

### Example 1

### Determination of the EphB4 Levels in Colorectal Tumours by Immunohistochemistry

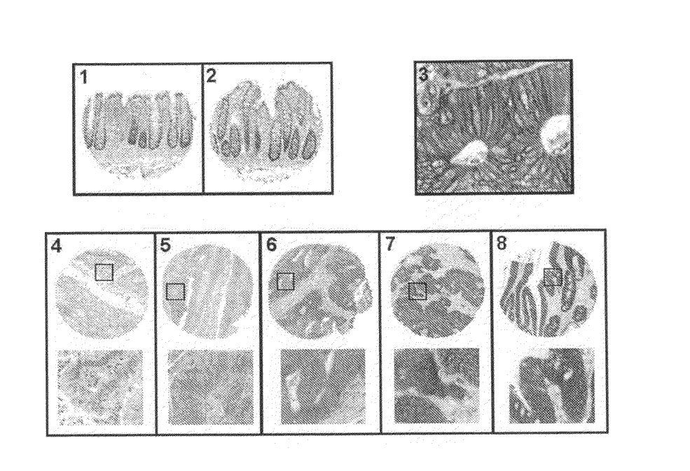

For these experiments a tissue microarray of paired tumour and normal colorectal samples of 137 patients was used. Each tissue was present in triplicate. The samples were collected in several medical institutes from the south of Finland, alter informed consent of all the patients. The expression levels of EPHB4 were determined by immunohistochemical staining with an antibody directed against the C-terminal region of human EPHB4 (Clone 3D7G8; Zymed Laboratories, San Francisco, Calif., USA). The specificity of this antibody has been demonstrated previously in formalin fixed in paraffin embedded tissues (Berclaz et al., (2003) Activation of the receptor protein tyrosine kinase EphB4 in endometrial hyperplasia and endometrial carcinoma. Ann Oncol, 14, 220-6). For the immunohistochemical staining, the commercial PowerVision Poly-HRP IHC (ImmunoVision Technologies, Brisbane, Calif., USA) kit was used according to the recommendations of the manufacturer. For the quantification of the expression level of EPHB4, semi-quantitative scale of 0 (absence of staining) to 4 (highest staining level encountered in the tissue matrix). As can be observed in FIG. 1, in the centre of the intestinal crypts of normal tissue expression level 4 is reached. For the study of a possible association between the tumour expression level of EPHB4 and survival, the mean of the 3 replicates in the matrix was used. For the analysis of survival, the Kaplan-Meyer representations and the Log-rank test were used, as were the regression model from Cox. When the level of EPHB4 was considered as a discrete variable, an expression below 2 was used as a threshold to define the group of patients with low expression levels of EPHB4 in their tumours.

### Example 2

### Determination of the EPHB4 Levels in Colorectal Tumours by Image Analysis after Immunohistological Staining

The same tissue microarray with paired samples employed in example 1; stained as described in this example, was analyzed by image analysis. All sections without irregularities (tissues en bad state) were quantified. In total, 86 tissue samples from patients with colorectal cancer were analyzed. Each of the samples was present in triplicate, and sections corresponding to tumour tissue were analyzed.

The images corresponding to each of the sections present in the microarray were acquired with an optical microscope using the AnalySIS, Soft Imaging System GMBH software. The image analysis was performed with the same software.

The original image was treated using the following filters: a DCE (Differential Contrast Enhancemente) contrast filter. The parameter for bandwidth was fixed at 60; enhancement at 40; Edge Enhance Filter, particle size was fixed at 3 pixels and enhancement at 30%; conversion to grey scale was applied and particles were filtered with a minimum size of 900 pixels, in order to exclude cell nuclei stained with the hematoxilin-eosine counterstain. The parameters measured were: fractionated area, mean integrated grey scale values in the region of interest; mean grey scale and variance of grey scale.

A macro was written to measure these variables semi-automatically. The macro used was the following:

```
‘open document
docRestore( );
Op.Activate( );
docMaximize( );
‘define analiysis area
DefineROIs$( );
‘DCE Filter
DefineDCEFilter$(File:=NULL);
DCEFilter(File:=NULL);
‘Edge filter
DefineEdgeEnhance(Size:=3, Percent:=30);
EdgeEnhance( );
‘Color separation
ColorSeparationIntensity( );
‘Detection parameters
SetGrayThresholds$(Thresholds:=NULL, AutoName:=NULL);
DefineDetection(ROIs:=1, Border:=ANA_BORDER_CUT,
  Inclusions:=TRUE, MinPixel:=900, Min:=0, Max:=DBL_MAX,
  Unit:=”μm”, Connectivity:=ANA_CON_INCLDIAGONALS,
  UseRanges:=TRUE);
‘Detection and copy results
Detect( );
FrameROIResults( );
shSetSelection (0, SH_SEL_ROW, 1);
Copy( );
Close(AskForSave:=TRUE);
```

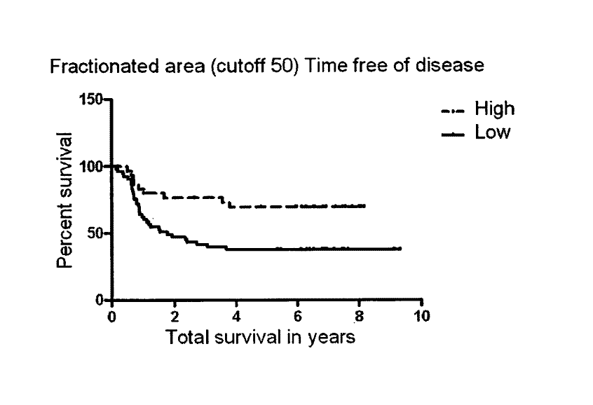

In FIG. 3; the mean of the three replicas in the matrix were used. To analyse the survival Kaplan-Meyer and the Log-rank (Mantel-Cox) Test representations were used. The level of EPHB4 was treated as a discrete variable. The group of patients with low levels of EPHB4 was defined as those tissues with a fractionated area below 50% (area by anti-EPHB4 antibody above the cutoff level with respect to total tumour area).

The quantification of the immunohistochemistry by image analysis gives a higher significance than the manual anatomopathological valoration, and confirms the pronostic value of EPHB4 levels with respect to the disease free time. (p=0.009 for the manual measurement and p=0,0065 for the automated measurement using the image analysis software.

### Example 3

### Determination of the Levels of EphB4 in Colorectal Cancer Cell Lines Using Western Blot

100 microgram fractions of protein extracts from the colorectal cancer cell lines were separated in a 7% SDS-polyacrylamide gel. The proteins were transferred to a nitrocellulose membrane and stained with an anti-EPHB4 antibody (dilution 1/200; Clone 3D7G8; Zymed Laboratories, San Francisco, Calif.) as described previously (Arango et al., (2003) c-Myc overexpression sensitizes colon cancer cells to camptothecin-induced apoptosis. British Journal of Cancer, 89, 1757-65). Afterwards, the membrane was stained with an anti-actine antibody (clone AC74, 1/1000; Sigma) to ensure equal loading in each lane (Arango et al., (2003) c-Myc overexpression sensitizes colon cancer cells to camptothecin-induced apoptosis. British Journal of Cancer, 89, 1757-65).

### Example 4

### Clonogenic Assay

Cultures of SW837 cells were transfected (Lipofectamine 2000; Invitrogen, Carlsbad, Calif., USA) with an expression vector of EPHB4 (pcDNA-EPHB4) or with the corresponding empty vector (pcDNA3.1, Invitrogen, Carlsbad, Calif., USA). After 24 h the cells were trypsinized and resawn in a dilution of 1/10. After 2 weeks of selection on 500 μg/ml neomycin (Invitrogen, Carlsbad, Calif., USA), surviving colonies were fixed in 4% paraformaldehyde and stained with crystal violet.

### Example 5

### Treatment of Colorectal Cancer Line

Cultures of the colorectal cancer cell line SW620, characterized by a low expression level and methylation the EphB4 promoter; were treated during 72 h with different concentrations of 5aza- cytidin. The cells responded with an increase of the level of gene expression, determined by RT-PCR, being 10 μM the most effective dose.

1. A method for arriving at a prognosis for colorectal cancer in a patient, the method comprising
determining an expression level of EphB4 gene in a biological sample isolated from the patient, in which the expression level of the EphB4 gene is used as a prognostic marker and as a marker for response of a colorectal tumour to treatment with 5-fluorouracil.

2. The method according to claim 1, where low expression levels of EphB4 gene are associated with a bad prognosis and a poor sensitivity of the colorectal tumour to treatment with 5 fluorouracil and high levels of expression are associated with a good prognosis and high sensitivity to said treatment.

3. The method according to claim 1, where the biological sample is isolated from cells obtained from biopsies or any other method of extraction from the patient.

4. The method according to claim 1, where the sample analyzed is RNA coded by the EphB4 gene or fragment thereof.

5. The method according to claim 4, where the determination is carried out by means of a method for nucleic acid amplification.

6. The method according to claim 4, where the determination is carried out by means of a microarray elaborated with deposited oligonucleotides.

7. The method according to claim 4, where the determination is carried out by means of a microarray elaborated with oligonucleotides synthesized in situ.

8. The method according to claim 4, where the determination is carried out by in situ hybridization using specific probes labelled through any labelling method.

9. The method according to claim 4, where the determination is carried out through gel electrophoresis.

10. The method according to claim 9, where determination is carried out through transfer to a membrane and hybridization with a specific probe.

11. The method according to claim 4, where the determination is carried out by a diagnostic imaging technique.

12. The method according to claim 4, where determination is carried out with detectable paramagnetic nanoparticles functionalized with antibodies.

13. The method according to claim 1, wherein the biological sample analyzed is a protein encoded by the EphB4 gene or fragment thereof.

14. The method according to claim 13, where the determination is carried out through incubation with a specific antibody.

15. The method according to claim 14, where the determination is carried out by Western blot.

16. (canceled)

17. The method according to claim 13, where the determination is carried out through gel electrophoresis.

18. The method according to claim 13, where the determination is carried out by protein chips.

19. The method according to claim 13, where the determination is carried out by means of ELISA.

20. The method according to claim 13, where the determination is carried out by a diagnostic imaging technique.

21. The method according to claim 13, where the determination is carried out by a diagnostic imaging technique using detectable nanoparticles functionalized with antibodies.

22. A kit to perform the method according to claim 1, that comprises reagents and additives to determine the expression level of EphB4.

23. A kit that comprises:
a primary specific anti-EPHB4 antibody,
a secondary antibody able to conjugate with the first antibody,
at least one visualization reagent,
a chromogenic substance, and
other reagents and additives useful to determine the expression level of EphB4 in a tissue section.

24. The kit according to claim 23, wherein the kit comprises positive and negative controls that permit quantification of expression levels of EphB4.

25. A method of treating colorectal cancer in a subject, the method comprising:
increasing the subject's expression levels of EphB4.

26. (canceled)

27. (canceled)
